(19) United States
(12) Patent Application Publication
(10) Pub. No.: US 2010/0143265 A1
(43) Pub. Date: Jun. 10, 2010

(54) SYSTEM AND METHOD FOR THE DELIVERY OF A SANITIZING FOAM

(75) Inventors: Leroy Hewes, Modesto, CA (US); Scott F. Unruh, Modesto, CA (US)

Correspondence Address: FLIESLER MEYER LLP, 650 CALIFORNIA STREET, 14TH FLOOR, SAN FRANCISCO, CA, 94108, US

(73) Assignee: GREEN SOURCE AUTOMATED, LLC, Ceres, CA (US)

(21) Appl. No.: 12/633,587

(22) Filed: Dec. 8, 2009

Related U.S. Application Data
(60) Provisional application No. 61/121,110, filed on Dec. 9, 2008.
(60) Provisional application No. 61/145,534, filed on Jan. 17, 2009.

(51) Int. Cl. A61K 9/12; A61K 33/18; A61P 31/00

(52) U.S. Cl. 424/43; 424/667

(57) ABSTRACT

A spray solution generates a foam to sanitize surfaces. The spray dip solution is a mixture of the solution with carbon dioxide and nitrous oxide in a closed vessel. After ejecting the liquid dip solution mixture from a nozzle a foam is generated from the solution as the solution hits a surface. The spray can be applied to a number of different surfaces. In an embodiment of the invention, the spray can be applied to cow teats without risk of cross-contamination of the nozzle or other equipment. The generated foam remains intact on the teat for a prolonged period of time, increases the area of the teat in contact with the foam during the treatment period and disperses skin-conditioning fats present in the dip treatment, thereby increasing the coverage of the skin conditioner on and within the epidermis of the teat and surrounding areas.

### CLAIM TO PRIORITY

The present application claims the benefit of priority under 35 U.S.C. §119(e) to U.S. Provisional Patent Application entitled “Spray Sanitizing Agent Spray-Foam,” Application No. 61/121,110, filed on Dec. 9, 2008, and 61/145,534, filed on Jan. 17, 2009, which applications are incorporated herein by reference in their entireties.

### FIELD OF THE INVENTION

This invention is in the general field of a preparation of a gas diffused spray. The gas diffused spray can be used for disinfecting surfaces as well as for fighting fires.

### BACKGROUND OF THE INVENTION

Disinfectants or sanitizers are antimicrobial agents that are applied to animal body parts, objects and surfaces to destroy microorganisms, the process of which is known as disinfecting or sanitizing. A disinfectant or disinfectant agent under an EPA definition kills 99.99% or more of specified bacteria. A sanitizer or sanitizing agent can be capable of killing 99.9%, of a specific bacterial test population, and do so within a specified period of time (sometimes 30 seconds). Sterilizing is the complete elimination of all microorganisms. These definitions can vary, but the effect is the reduction of bacteria and pathogens.

The main difference between a sanitizer and a disinfectant is that at a specified use dilution, the disinfectant can have a higher kill capability for pathogenic bacteria compared to that of a sanitizer. Few disinfectants and sanitizers can sterilize and those that can depend entirely on their mode of application. Bacterial endospores are most resistant to disinfectants and sanitizing agents, however some viruses and bacteria also possess tolerance to disinfectants and sanitizing agents.

### Alcohols

Ethanol or isopropanol are sometimes used as a sanitizing agent or disinfectant, as they have wide microbiocidal activity and are non-corrosive. They also have limited residual activity due to evaporation and have reduced activity in the presence of organic material. Alcohols are more effective combined with purified water because the water content allows for greater diffusion through cell membranes. Alcohol is not effective against resistant fungal and bacterial spores:

### Aldehydes

Aldehydes, such as Glutaraldehyde, have a wide microbiocidal activity and are sporocidal and fungicidal. They are partly inactivated by organic matter and have slight residual activity.

### Oxidizing Agents

Oxidizing agents act by oxidizing the cell membrane of microorganisms, which results in a loss of structure and leads to cell lysis and death. A large number of disinfectants and sanitizing agents operate in this manner. A sodium hypochlorite solution can be used to disinfect drains, toilets, and other surfaces. Hypochlorites yield an aqueous solution of hypochlorous acid that is the disinfectant. Hypobromite solutions can also be used. Sodium chlorite, sodium chlorate, and potassium chlorate are used as precursors for generating chlorine dioxide, an advanced disinfectant for drinking water to reduce waterborne diseases. Hydrogen peroxide is used in hospitals to disinfect surfaces and in the food packaging industry to disinfect foil containers. Peracetic acid is a disinfectant produced by reacting hydrogen peroxide with acetic acid. It is broadly effective against microorganisms and is not deactivated by catalase and peroxidase, the enzymes that break down hydrogen peroxide. Performic acid produced by reacting hydrogen peroxide with formic acid, reacts more rapidly and powerfully than peracetic acid before breaking down to water and carbon dioxide. Iodine is usually dissolved in an organic solvent or as Lugol's iodine solution (a solution of elemental iodine and potassium iodide in water). Iodine is sometimes used after forming an iodine complex (complexed iodine) and often in combination with skin protecting ingredients, such as glycerine. It is used in the poultry and dairy industries. Ozone is a gas that can be added to water for sanitation. A potassium permanganate (KMnO₄) solution can be applied to wounds in dilute solution. Potassium peroxymonosulfate is a wide-spectrum disinfectant that kills bacteria, viruses, and fungi.

### Phenolics

Phenol and o-phenylphenol are active ingredients in some household disinfectants, mouthwashes and hand washes. Chloroxylenol is a common household disinfectant and antiseptic. Thymol, derived from the herb thyme, is the active ingredient in a disinfectant.

### Quaternary Ammonium Compounds

Benzalkonium chloride has been used as low level disinfectants and is effective against algae, bacteria and enveloped viruses, but not against some species of Pseudomonas bacteria or bacterial spores.

### Other

Polyaminopropyl biguanide is specifically bactericidal at very low concentrations. It has a unique method of action: the polymer strands are incorporated into the bacterial cell wall, which disrupts the membrane and reduces its permeability, which has a lethal effect to bacteria. It is also known to bind to bacterial DNA, alter its transcription, and cause lethal DNA damage. Acetone is used in the meat processing industry as a sanitizing agent. High-intensity shortwave ultraviolet light can be used for disinfecting smooth surfaces such as dental tools, but not porous materials that are opaque to the tight such as wood or foam.

### Uses for Disinfectants and Sanitizers

Disinfectants and sanitizers are routinely used in, among other places, food processing facilities (e.g., facilities for processing of beef, poultry, ice cream, soft drinks; dairies), food service facilities (e.g., restaurants, cafeterias), healthcare facilities (e.g., hospitals, surgery centers, clinics, long-term care), hospitality facilities (e.g., hotels, motels, resorts), educational facilities (e.g., schools, universities), and commercial facilities (e.g., office buildings, shopping malls, retail stores).

When applied to inanimate or animate surfaces in those various settings, the disinfectants and sanitizers should be applied to the surfaces uniformly and remain on the surfaces for an appropriate length of time necessary to destroy, inhibit, and/or prevent the growth of microorganisms. Nevertheless, most liquid-based disinfectants and sanitizers are not applied to the surfaces in a manner that supports optimal environmental disinfection.

### Dairy Industry

In the dairy industry a variety of “teat dip” solutions can be used to treat cows either before or after the milking operation to help inhibit the spread of pathogens. For example, a cow's teats can be dipped in a teat dip solution before and after milking occurs. Pre-milking teat dipping sanitizes the teats before the teats have expanded to allow the flow of milk through the teat's orifices. Pre-milking teat dipping is one of the most effective management practices to prevent environmental or clinical mastitis.

After milking and before the teat orifice has closed, a cow can come into contact with pathogens. Thus, post-milking teat dipping can help insure that a pathogen that comes into contact with the expanded teat is inactivated and thereby cannot cause a bacterial infection. Post-milking teat dipping is also used to help insure that the milking equipment remains sterile.

### Dip Solution in Cup

A teat dip solution generally contains a sanitizing agent, varying amounts of skin conditioners (fats and oils) and water. The ingredients are blended and applied to sanitize the teats of milk cows and insure that no pathogen is transmitted between cows during the milking procedure. The dip solution can be directed into a cup and then the cup raised to immerse the teat of the cow in the dip solution. Efficacy of the dip solution is determined by the ability of a teat dip solution to prevent new intra-mammary infections by mastitis pathogens. Iodine is a common sanitizing agent in teat dips. Other ingredients are added to the teat dip in order to improve the skin condition of the cow udder and teats that are exposed and manipulated during the milking process.

### SUMMARY OF THE INVENTION

In an embodiment of the invention, a gas is diffused into a sanitizing or disinfecting solution and used to form a liquid spray that generates a foam on the surface to be treated. The gas can be a single gas, a mixture of water soluble gasses, a mixture of fat/oil soluble gases, and/or a gas that has a high solubility in the desired solution. The sanitizing solution can include water, conditioners (e.g., fats, oils), and/or a sanitizing agent (e.g., iodine). In an embodiment of the invention, the gas is a mixture of carbon dioxide and nitrous oxide. The gas mixture containing carbon dioxide and nitrous oxide gasses, which have been dissolved into the sanitizing solution, can also be used as a propellant for the preparation.

In an embodiment, the preparation can be a teat dipping solution applied to a cow's teats. The preparation formed is stable and can be dispersed as an aqueous solution through a nozzle. The solution, with the gas dissolved therein, exits the dispersing nozzle and foam is formed on the target surface. In an embodiment of the invention, the preparation can be directly sprayed on the teat and in this manner avoid the risk of cross contamination between the cows. In an alternative embodiment of the invention, the foam produced from the preparation can be produced in a cup or other kind of container and then applied to a cow's teats. In an embodiment of the invention, the foam produced from the preparation can be applied by a manual method to a cow's teats. In various embodiments of the invention, the foam can be applied by automated methods of dipping.

The applied foam remains intact on the teat for a prolonged period of time compared to dipping the teat in an aqueous dip solution, thereby increasing the length of time of exposure of the teat to the sanitizing agent. In an embodiment of the invention, a propellant/diffused gas mixture of carbon dioxide and nitrous oxide, results in relatively small bubble size of gas components within the foam compared to the bubble size in a foam formed with carbon dioxide as the propellant/diffused gas. In an embodiment of the invention, a propellant/diffused gas mixture of carbon dioxide and nitrous oxide results in relatively small bubble size of gas components within, the foam compared to the bubble size in a foam formed with air as propellant. The small bubble size of the foam increases the area of the dip solution in contact with the teat compared to foam with larger bubbles. The use of nitrous oxide in the foam disperses the skin conditioning fats present in the dip treatment thereby increasing the coverage of the skin conditioner on and within the epidermis of the teat and surrounding areas.

In an embodiment, the foam can be applied to and sanitize or disinfect other objects and surfaces aside from cows and other living animals. By way of example only, these objects and surfaces may be found in food processing facilities (e.g., facilities for processing of beef, poultry, ice cream, soft drinks), food service facilities (e.g., restaurants, cafeterias), healthcare facilities (e.g., hospitals, surgery centers, clinics, long-term care), hospitality facilities (e.g., hotels, motels, resorts), educational facilities (e.g., schools, universities), and commercial facilities (e.g., office buildings, shopping malls, retail stores).

Embodiments of the invention are not limited to disinfecting and sanitizing surfaces. In other embodiments, the preparation can be used fire fighting foam.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a graph of the vapor pressure versus temperature of CO₂;

FIG. 2 shows a graph of the vapor pressure versus temperature of N₂O;

FIG. 3 shows a schematic of an embodiment of a system in accordance with this invention;

FIG. 4 shows a picture of a 50% carbon dioxide and 50% nitrous oxide propellant/diffused gas preparation directed onto a human finger in accordance with an embodiment of the invention;

FIG. 5 shows a picture of a 50% carbon dioxide and 50% nitrous oxide propellant/diffused gas preparation directed onto a cow's teat in accordance with an embodiment of the invention;

FIG. 6 shows a picture of a liquid spray solution directed onto a leather glove;

FIG. 7 shows a picture of a 100% carbon dioxide propellant/diffused gas preparation directed onto a leather glove in accordance with an embodiment of the invention;

FIG. 8 shows a picture of a 100% nitrous oxide propellant/diffused gas preparation directed onto a leather glove in accordance with an embodiment of the invention;

FIG. 9 shows a picture of a 90% carbon dioxide and 10% nitrous oxide propellant/diffused gas preparation directed onto a leather glove in accordance with an embodiment of the invention;

FIG. 10 shows a picture of a 50% carbon dioxide and 50% nitrous oxide propellant/diffused gas preparation directed onto a leather glove in accordance with an embodiment of the invention; and

FIG. 11 shows a picture of a 10% carbon dioxide and 90% nitrous oxide propellant/diffused gas preparation directed onto a leather glove in accordance with an embodiment of the invention.

FIG. 12 shows a picture of air aspirated sanitizing preparation manually applied to a leather glove using a dip cup in accordance with an embodiment of the invention.

FIG. 13 shows a picture of 50% carbon dioxide and 50% nitrous oxide preparation manually applied as a foam to a leather glove using a dip cup in accordance with an embodiment of the invention.

FIG. 14 shows a block diagram of a apparatus having charging stations engaged to storage tanks in accordance with an embodiment of the invention.

FIG. 15 shows a cross-sectional view of an aerosol can in accordance with an embodiment of the invention.

FIG. 16 shows a heat source being applied to a surface to promote foaming in accordance with an embodiment of the invention.

FIG. 17 shows a heat source being applied to the preparation to promote foaming in accordance with an embodiment of the invention.

### DETAILED DESCRIPTION OF THE INVENTION

### Dip Solutions in the Dairy Industry

A wide variety of dip solutions are used in the diary industry. For example, teat dip solutions are sold by Ecolab under the trade names of Legend and Optima and by DeLaval under the trade names of Della Care, Della Soft, Della One, Della One-Ten, and Triumph. These dip solutions are typically aqueous solutions that include a sanitizing agent and varying amounts of skin conditioners (i.e., emollients). A dip solution may have 1-5% sanitizing agent, 2%-15% skin conditioners (e.g., glycerin), and the rest water. Sanitizing agents in dip solutions may include, but are not limited to, iodine, acidified sodium chlorite, sodium hypochlorite, chlorhexidine, hydrogen peroxide, caprylic/capric acid(s), bronopol, dodecyl benzene sulfonic acid (DDBSA), nisin, glycerol monolaurate, and quaternary ammonium.

An example of a chlorine based dip would be Valient produced by ABS. Two part dips that must be combined before application can also be used. For example, part one of a two part dip may include lactic acid 2.3%, linear dodecyl, and benzene sulfonic acid (emoliant system), where part two includes sodium salt of chlorous acid. Combining the salt of part two with the acid of part one creates a reaction that produces active chlorine. This particular mix would have a limited shelf life after mixing.

The teat dip solution can be used to disinfect a cow's teats before and after the cow has been milked. The dip solution can be applied by using manual or robotic methods. Preparation composition coverage and contact time are the qualities that generally determine the bacteriological kill rate of the dip solution. The longer the dip solution can stay in contact with the teat the greater the sanitization of the teat.

Liquid Dip Solution v. Air Aspirated Foam Dip Solutions

Liquid dip solutions can readily be applied to a cow's teats using, among other things, robotic spraying procedures. By implementing a liquid spraying procedure, good coverage and accurate application on the teats can be accomplished. Nevertheless, a problem with using a liquid dip solution is that the contact time of the liquid dip solution on the teats is limited. Put simply, the liquid tends to run off the teats, thereby limiting contact time.

To increase contact time, air can be injected into a liquid dip solution to create a foam-based dip solution. It was experimentally determined that foam-based dip solutions can remain on a cow's teats for a greater amount of time than liquid-based dip solutions since the foam-based solution will not immediately run off a cow's teats like a liquid-based solution. Rather, it can remain on the cow's teats until it drips off the teat or dries on the teat or can remain a specified time on the cow's teats and can then be removed either by applying a stream of gas to blow the foam off the teat or by wiping the foam off the teat with a sterile cloth. Moreover, air aspirated dip solutions were found to require less dip solution for the same coverage when compared to liquid dip solutions (which will be described in greater detail below). Using less dip solution to provide the same coverage is particularly advantageous in the dairy industry given the high cost of dip solutions.

Despite its advantages, a problem with using an air aspirated foam dip solution is that it can be difficult to apply on a cow's teats. For example, one method that can be used to apply the foam-based dip solution is to generate the foam, and then place the foam into a cup or vessel that can then be transported to the teat location by a robotic system. This method will deliver the dip solution and provide the necessary coverage, but also has a number of drawbacks. One drawback is that this method slows down the speed with which the robotic system can deliver the dip solution compared to a liquid spray system. Another drawback is that it is difficult to assure that the cup or vessel has been properly cleaned between uses so as to eliminate any cross contamination between two or more cows.

Pre-created air aspirated foam can also be applied using a nozzle. In this situation, the nozzle does not make contact with the animal, nor does it carry any residual material that comes in contact with the animal. This helps to prevent cross contamination. However, it takes energy to get the foam to the cow teat from the nozzle. Thus, the foam must be accelerated to a sufficient velocity as to cause the foam to travel to the target. But when foam is accelerated it compresses and the foam bubbles break down giving up the trapped gas to the atmosphere. Further, air resistance causes the foam to lose its ability to maintain a tight cross-sectional area. Thus, the foam that survives is broken into many small “chunks” that because of their odd shapes fly in different paths in the general direction of the cow's teat, but no longer at a specific target.

Another drawback of using air aspirated foam is that it does not have the expansion capability (foaming agents) needed to form dense enough foam when using air as the propellant. The air aspirated foam forms large bubbles. Foam formed with large bubbles tend to leave fines lines of sanitizing agent and conditioner that follow the intersection points of the bubbles resulting in uneven and incomplete coverage of the teat after the bubbles have collapsed. Accordingly, large bubbles reduce the effectiveness of teat coverage with the dip solution, and thereby the effectiveness of the sanitizing procedure.

### Gas Diffused Dip Solution

As will be shown and described in greater detail below and in accordance with an embodiment of the invention, gas diffused dip solutions that generate a foam have been developed that can be applied with the ease of liquid dip solutions, while also being more effective than air aspirated foam dip solutions. In an embodiment of the invention, a pressurized mixture of carbon dioxide and nitrous oxide can be dissolved into a teat dip solution in a suitable vessel designed for this purpose. In the vessel, the teat dip solution reaches a saturation equilibrium with the gases and a gas/liquid interface forms. The gas or gasses are diffusible gasses that can, at least partially, dissolve in the aqueous teat dip solution and the gas or gasses can include a water soluble component and a component that solvates with the skin conditioner provided in the teat dip solution. The gas space still at pressure provides the motive force or gas propellant for the application of the dip/gas mixture. Constant pressure is maintained by the addition of gas from a regulated source and as the dip solution is applied the level of dip solution can be maintained by the automatic addition of dip solution from an atmospheric storage vessel by means of an appropriate pumping system. The dip/gas mixture or preparation is directed at the teat of the cow by a specially designed spray nozzle. The nozzle is constructed so that a shut off mechanism is located as close to the spray tip as possible. This shut off mechanism is remotely operated by electric, pneumatic or other means as necessary for the specific application. When this remotely controlled spray valve is actuated the dip/gas mixture or preparation can be discharged, in a specific pattern determined by the orifice in the spray nozzle, at the target, the cow's teat. As the dip/gas mixture or preparation passes across the orifice in the spray nozzle, the preparation travels from an area of above atmospheric pressure to atmospheric pressure.

### Gas Characteristics

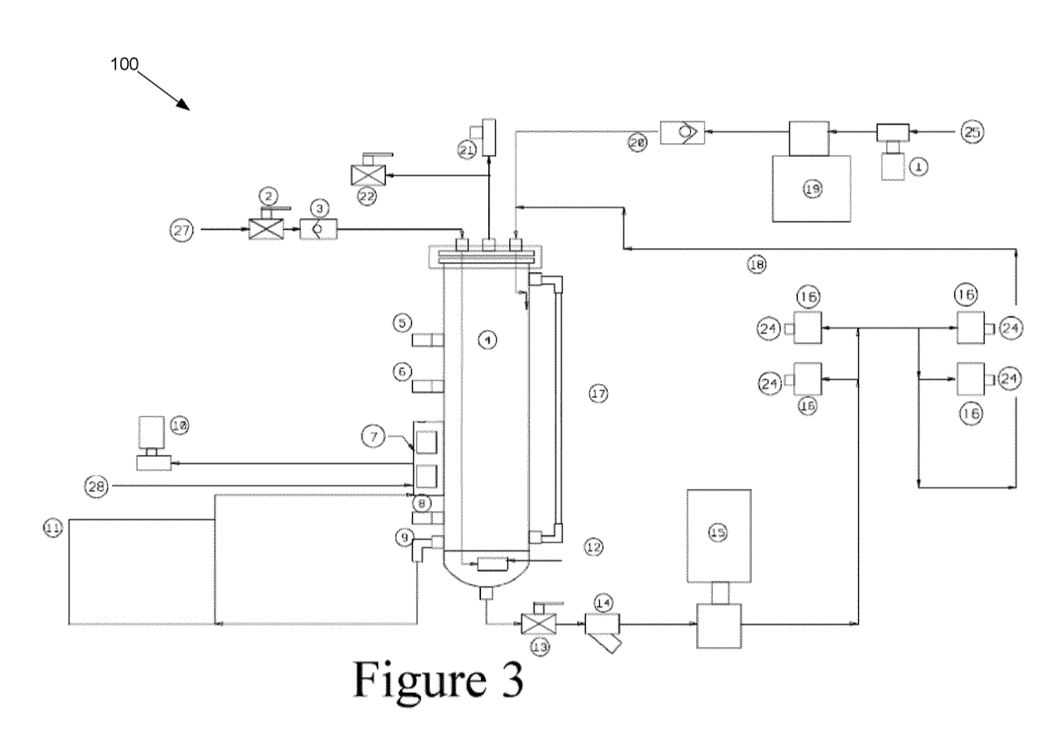

It is desirable to store the gas or gasses used in the spray system (FIG. 3) in liquid form. The reason to use gasses stored in liquid phase is that a 1 cu. ft. tank would hold approximately 8 cu. ft. of usable gas in liquid form as opposed to the highest pressure allowed in a gaseous state. Consider a tri-mix welding gas delivered with the combination of CO₂, N₂ and He in a 1 cubic foot (cu. ft.) cylinder. When the gas supplier fills the tank it is pressurized. The higher the pressure the more gas that there is in the tank to use. The gas supplier has to stay below the pressure of the gas in the tri-mix with the lowest vapor pressure. Vapor pressure is the pressure of a vapor in equilibrium with its non-vapor phase. If the pressure of the tri-mix cylinder were increased to a pressure higher than one or more of the gasses vapor pressure values, those gasses would change state and sit at the bottom of the tank in liquid form. As the gas was used from the tank the “mix” of gasses would no longer be in the same percentage as the tank had at a pressure above the vapor pressure of the gasses. If the pressure of the tank exceeded all of the tri mix gasses vapor pressures all of the gasses would be in liquid form, but as the gasses were used, the highest percentage of gas coming from the tank would be the gas with lowest vapor pressure and so forth (in order of the vapor pressures of the individual gasses from lowest to highest).

Using multiple cylinders, one gas per cylinder, would obviate the problem with mixing gasses in liquid form, but it would also multiply the shipping, installed base cost and add the cost of a mechanism to meter the proper amounts of each gas to get the required blend.

The combination of CO₂ and N₂O is unique because the vapor pressure of the two gasses are almost identical. Gas suppliers do not sell the combination of CO₂ and N₂O in liquid form for use of these gasses in embodiments of the present invention. CO₂ and N₂O can be combined in any combination of percentages in liquid form and when the liquid is allowed to turn into a gas, the percentage of the two gasses remains relatively the same as the percentages in the liquid state.

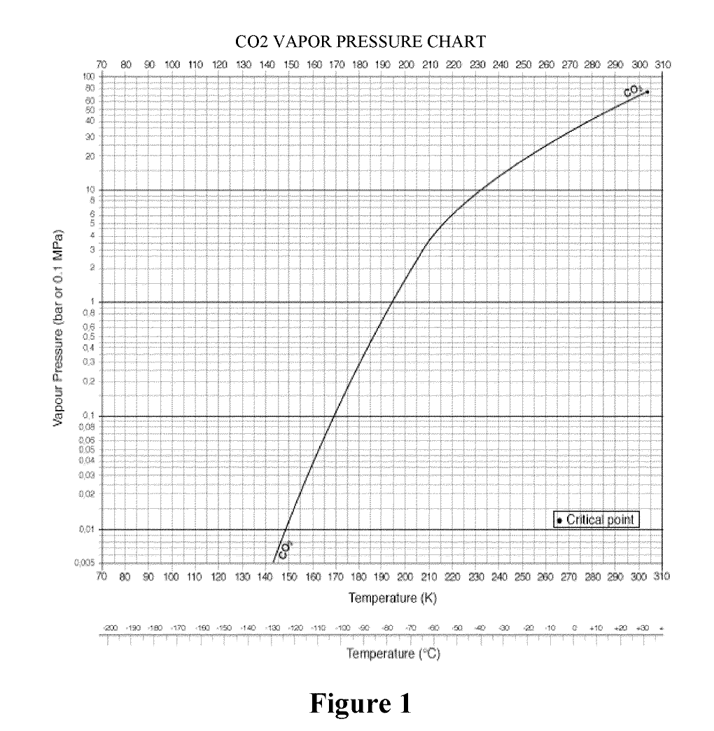

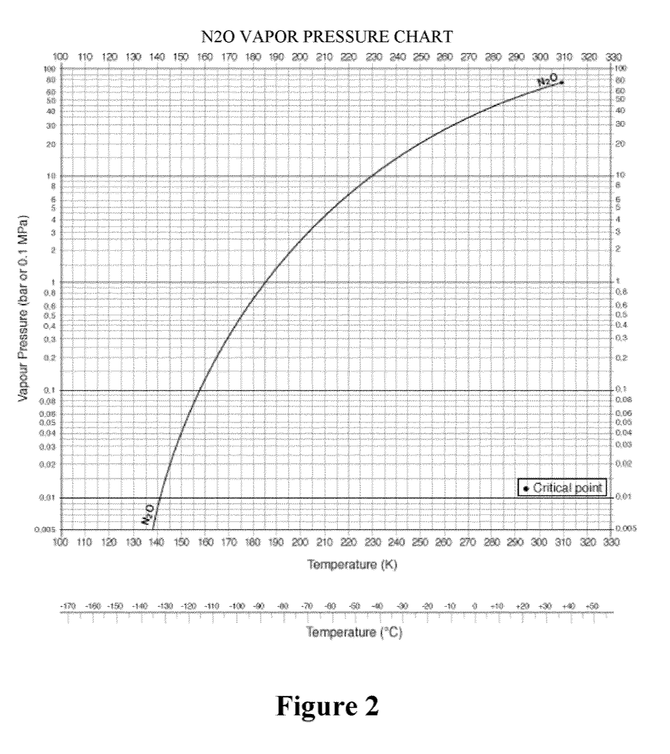

Both CO₂ and N₂O gasses can be easily distributed, stored and used in liquid form. This is beneficial because as stated below, a liquid storage vessel can hold a great deal more gas vapor (liquid converted to vapor) than the same container with compressed gas alone. However, generally when storing two gasses in a liquid form, the resulting availability of the two gases as vapors depends on the relative vapor pressure of each gas. Generally, most gas combinations when held in liquid form do not vaporize at the same percentage as the liquid mix. The gas with the lower vapor pressure will vaporize in the greatest quantity. In order to compensate, compressed gas cylinders can be used that hold a fraction of the product when compared to a liquefied gas storage system. This results in greater transportation and handling cost. Alternatively, the two gasses can be held in separate vessels in liquid form. But this increases the bulkiness of the equipment and results in increased storage space, freight costs and the need to use mixing equipment. To illustrate this point, CO₂ has a vapor pressure at 290° K. of approximately 50 (bar), while N₂O also has a vapor pressure at 290° K. of approximately 50 bar (see FIG. 1 and FIG. 2). At 185° K., CO₂ has a vapor pressure of approximately 0.45 bar, while N₂O has a vapor pressure at 185° K. of approximately 1 bar. Thus, a 50% N₂O and 50% CO₂ liquefied mixture vaporizes to give approximately 50% N₂O and approximately 50% CO₂.

When a vessel containing the dip solution is pressurized, the gas mixture pressure causes the gasses to dissolve into the dip and an amount of energy caused by the pressurization acts upon the system. The system is allowed to come to equilibrium and the temperature is controlled such that any temperature rise caused by this energy introduction is removed. Henry's law states: “the amount of any given gas that will dissolve in a liquid at a given temperature is a function of the partial pressure of the gas that is in contact with the liquid and the solubility coefficient of the gas in the particular liquid.” In other words, the amount of gas that can be dissolved into a liquid depends on the temperature, the pressure, and how well the particular liquid absorbs gases. With that being said, in order to make a consistent application of the preparation as a foam on the teats of a cow, a consistent pressure and temperature is beneficial. A recirculation loop from the pressurized tank to the nozzle and back to the tank can, for example, be employed to make sure the temperature and dissolved gas content remain consistent with that of the dip mixture in the tank in an embodiment of the invention.

A tank being maintained at constant pressure represents storage of energy. Opening the spray nozzle converts that stored energy into a mass at velocity, where the velocity is given by equation (1).

E=mv² equation (1),

where E represents energy, m represents mass and v represents velocity

The energy propellant force applied to the preparation, is what forces the dip solution preparation to project towards the cow's teat. The resulting pressure drop going across the nozzle causes the dip/gas mixture to begin to expand. But the preparation does not instantly expand to its full potential like popcorn when it pops. The expansion causes the mixture to cool and this slows the expansion (as dictated by Henry's law) as the mixture of gas and dip solution re-equilibrate. The cooling limits the cross-sectional area expansion of the sprayed dip, thereby limiting the reduction in velocity (which would be caused by friction from the liquid traveling through the air). The reduced cross-sectional area results in more liquid hitting the target. The remaining energy contained in the dissolved gas is liberated when the spray contacts the warm teat, and the energy release forms the foam on the teat as the dissolved gas is release from the sanitizing liquid teat dip preparation.

### Implementation

In various embodiments of the invention, a pressure vessel is used to hold the dip solution and expose it to the propellant/diffused gas or gasses. The pressure vessel was constructed from a 0.1 m outside diameter piece of stainless steel tubing. The ends of the tube were capped with tri clover fittings to allow ease of access. The tri-clover caps were modified by adding two entry points on the proximal cap. One entry point on the proximal cap is to allow propellant/diffused gas to be introduced and the other is used to bleed off atmospheric air, when the tank was initially filled with propellant/diffused gas and to relieve pressure before refilling. On the distal cap of the tank two openings were added, each with tubing securely attached and each tube going to a manual shut off valve with a spray nozzle attached. A propellant/diffusion gas source with regulator was attached to the pressure vessel through the entry point on the proximal cap. The pressure vessel is filled about half full with teat dip solution, closed and the tank is pressurized with approximately 100 pounds per square inch (psi) of propellant/diffusion gas, except for the 50% carbon dioxide and 50% nitrous oxide propellant/diffusion gas which is set at 150 psi. The distance and angle from nozzle to teat is 0.15 m and 45 degrees from the horizontal (which were set to mimic a robotic tool used for some embodiments). By directing the preparation from beneath the cow's teat and from two (a forward and a backward) directions, the preparation can contact the entire bottom and side surfaces of the teat.

In earlier embodiments of the invention, prior to positioning the spray valve immediately before the dispensing nozzle or orifice, it was positioned a distance from the dispensing nozzle. In this preliminary configuration, it was found that the gas diffused dip solution dribbled out of the dispensing nozzle after the spray valves were closed. Typically, a solution can be held in position in tubing by vacuum or capillary action. Since the gas diffused dip solution cannot form a vacuum this can explain why the gas diffused dip solution is not retained inside the tube. Alternatively, the gas diffused dip solution or preparation may not form sufficiently strong intermolecular bonds with the tube surface to generate capillary action and therefore the gas diffused dip solution is not retained inside the tube.

In an earlier embodiment of the invention, the apparatus did not recirculate the dip solution stored before the spray valves. In this preliminary configuration, the characteristics of the foam as applied to the animal were found to be irreproducible. This was attributed to variations in either the temperature of the dip solution occurring when the diffused dip solution was stored before the spray valve, and/or variations in the volume of gas dissolved in the dip-solution when the dip solution remained for different lengths of time in the connections prior to expulsion of the dip solution upon the spray valve opening.

FIG. 3 illustrates a schematic of an embodiment of a spray system 100 or of the invention. As shown in FIG. 3, teat dip solution 25 enters the apparatus through manual shut off valve 1. Supply pump 19 adds sufficient pressure to the liquid to overcome the pressure in the tank opening check valve 20 and allowing teat dip solution 25 from the dip supply tank (not shown) to fill the pressurized vessel 4. Dip solution 25 is automatically added until the high-level switch 5 is activated. Additional dip solution 25 is added whenever the level drops to the operational level switch 6. If no dip solution 25 is available the system is shut down and an alarm is sounded if the dip level drops below the low level switch 8. The regulated gas supply 27 enters the system through a manual shutoff valve 2 and the gas passes through check valve 3 and is added as needed to maintain the operational pressure through a gas diffuser 12 located below the liquid level. A level indicating sight glass 17 is provided for visual confirmation of system operation. The vessel is temperature controlled by a control loop consisting of a temperature sensor 9 that sends a signal to a temperature controller 11. The controller, in turn, controls the activity of a thermoelectric heat pump 7 and the heat pump uses water source 10 to pull heat from the source and to dissipate heat to the solution or preparation in vessel 4. The gas diffused dip is delivered to the point of use by means of a recirculation loop 18. The purpose of the loop 18 is to ensure that the preparation being applied is at all times consistent in both temperature and gas volume with that of the dip preparation held in vessel 4. The dip temperature for the embodiments reported was 12° C. Thus, the recirculation loop 18 ensures that the foam formed from the sprayed dip solution is reproducible in all its physical and chemical characteristics. Dip passes through a manual shutoff valve 13 and ‘Y’ strainer 14 to remove particulate matter. Recirculation pump 15 provides the motive force to send the gas-diffused dip around the recirculation loop 18. In addition, in FIG. 3, with respect to vessel 4, water line 28 provides water to vessel 4 as required, and pressure relief valve 21 and manual pressure relief valve 22 are use to provide pressure relief to vessel 4 as required.

In an embodiment of the present invention, spray valves 24 are associated with the dispensing nozzle 16. The nozzle is a short orifice that extends beyond the valve and shapes the spray that come out when the valve is opened. In a preferred embodiment of the invention, the nozzle is as short as possible so that when the valve is closed these is very little gas diffused preparation that can leak from the nozzle and be wasted. Four spray valves 24 and nozzles 16 are typically used on a robotic post dip station, but this could be any number. In alternative embodiments of the invention, any sort of dispensing valve can be used either robotically controlled or by manual applicator. The recirculation loop enters the vessel 4 at the same point the dip supply enters. The dip is directed at the inside wall of the vessel 4 above the liquid level so as to maximize the surface area of the dip exposed to the gas thereby optimizing the gas diffusion into the dip.

The spray valve 24 is opened for approximately 0.1 seconds to allow approximately 120 mL of gas diffused dip solution to exit the nozzle 16. The preparation travels the approximately 0.15 m distance to the cow's teat largely as a stream of liquid with a small amount of foaming. Because the mechanical valve shuts off the orifice, no dripping of the teat solution from the nozzle occurs. Once the spray contacts the teat, the heat transfer from the animal allows the trapped gases to escape the liquid preparation and generate significant foaming.

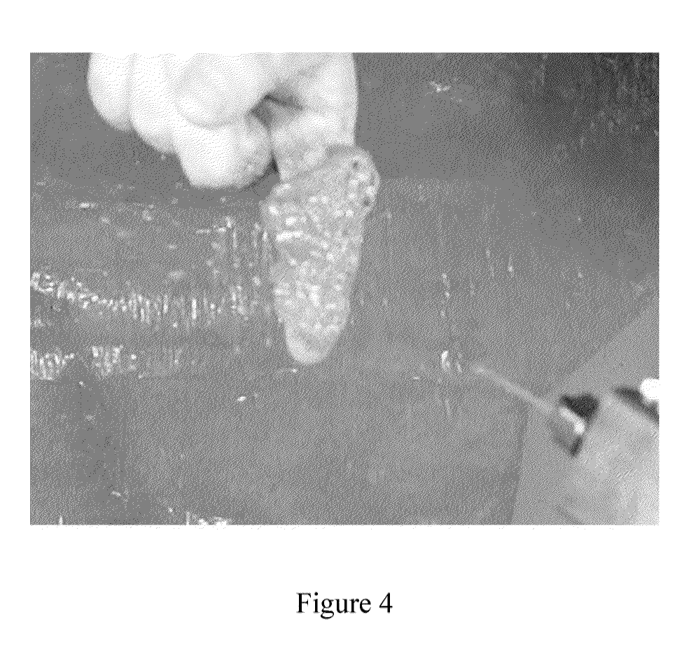

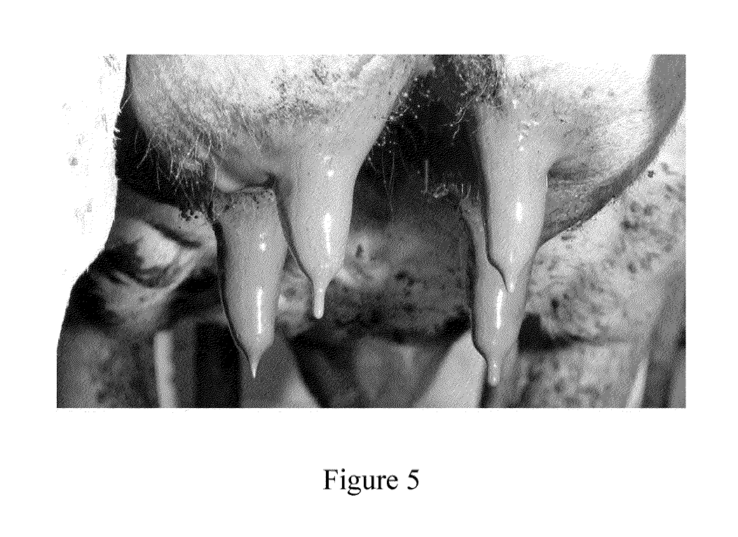

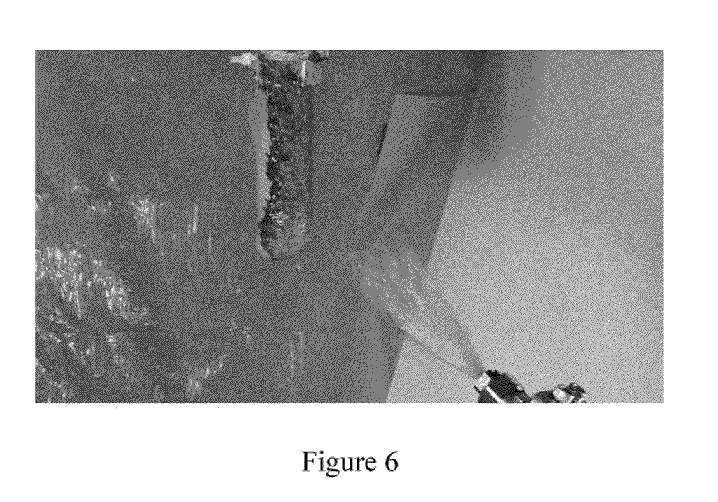

In order to simulate the cow's teat, measurements were carried out where a spray was directed at a human finger, as shown in FIG. 4. These measurements revealed that it was difficult to hold the finger stationery for long periods of time, and that the spray did not accumulate on the finger in the same manner as it accumulated on a cow's teat (compare FIG. 4 and FIG. 5). This was possibly due to the movement of the finger or alternatively to extraneous compounds present on the finger (such as lipids) which were either not present or not as abundant on the cow teat. Accordingly, in order to simulate the cow's teat, measurements were carried out where a spray was directed at a fixture simulation that included a leather glove finger provided over an aluminum cigar tube, with 120° C. water tilled inside the cigar tube. The spray included a dip solution manufactured by Westfalia Surge, which is sold under the trade name TeatKote 10/111. This particular teat dip includes approximately 1% titratable iodine, 89% water and 10% emollient (skin conditioners). This set-up was used for Examples 1 through 6, and FIGS. 6 through 13. Approximately the same experimental set-up with a 50% carbon dioxide and 50% nitrous oxide propellant/diffusion gas, but directed at a human finger is shown in FIG. 4. Approximately the same set-up with a 50% carbon dioxide and 50% nitrous oxide propellant/diffusion gas, but directed at cow's teats, is shown in FIG. 5.

In an embodiment of the invention, a cow teat's can be sanitized prior to milking. In this embodiment, the preparation can remain on the cow's teats until it drips off the teat or dries on the teat or can remain a specified time on the cow's teats and can then be removed either by applying a stream of gas to blow the foam off the teat or by wiping the foam off the teat with a sterile cloth.

In an alternative embodiment of the invention, a-cow teat's can be sanitized after milking. In an embodiment of the invention, the foam, can stay on the teat until the foam dries on the teat (see FIG. 5). In an alternative embodiment of the invention, the foam, can stay on the teat until the liquid dries on the teat. In another embodiment, the foam can remain on the cow's teats until it drips off the teat, comes in contact with another surface and coalesces off the teat, or dries on the teat. In an alternative embodiment of the invention, the foam can stay on the teat for up to approximately 10 to 15 minutes before the foam coalesces to form a liquid which stays on the teat until the liquid dries on the teat. Since the time the foam remains on the teat is longer than the time a liquid stays on the teat, the time of contact of the dip solution on the teat can be significantly increased by using a foam application. Further, the increase in volume of the foam due to foaming on contact with the warm object can result in greater coverage of the object. In addition, much less dip can be required for the same amount of coverage with longer contact time and thereby more effective sanitizing.

### Example 1

### Liquid Spray

Tests using the liquid spray delivery (FIG. 6) vs. the foam showed a reduction in dip usage for comparable teat coverage. When the liquid spray valve opening time was set at 0.2 second the coverage on the gloved finger with the liquid spray matched coverage on the gloved finger with the CO₂/N₂O foam and a 0.1 second spray valve opening time. However, by opening the spray valve for twice as long for the liquid spray much more (approximately 266 mL) dip solution was used compared to the 120 ml used to create the foam. The resulting usage of dip solution with the liquid-spray method exceeded the amount used for spray that resulted in foam, or the foam in the cup method. The majority of the liquid on the gloved finger had dripped from the teat within 5 seconds. FIG. 6 shows a picture of liquid spray directed onto a gloved finger. In this figure, air is used as the motive force to drive the dip through the nozzle to the teat.

It is noted that when the air propellant spray valve opening time was set at 0.1 seconds, the liquid spray did not result in significant coverage of the gloved finger. The spray valve applied approximately 133 mL of dip solution. In comparison, using the gas diffused (e.g., CO₂ see Example 2) dip, approximately 120 mL of dip solution was applied. This is due to the impact of the viscosity change after the dip solution is past the nozzle restriction.

### Gas Diffused Examples

The equipment (FIG. 3) was used with other propellant/diffusion gasses. In various embodiments of the invention, one or more other gasses can be introduced as a propellant/diffusion resulting in the formation of the foam.

### Use of CO₂ to Generate Foam

Carbon dioxide (CO₂) is soluble in water up to 1.45 g/L at 25° C. and 100 kPa. The amount of CO₂ that can be dissolved into the dip solution is dependent on the temperature and pressure of the solution. The lower the temperature of the solution the less pressure that is required to saturate the solution with CO₂. Conversely, the higher the temperature of the dip solution the more pressure that is required to saturate the solution with CO₂. It was found that at standard room temperature and 100 psi pressure conditions approximately 4.5 volumes of dip solution can be dissolved per volume of CO₂.

When the carbonated dip solution is released through a spray nozzle, the dip solution begins to effervesce as soon as it crosses the pressure drop at the mechanical valve feeding the nozzle. The resulting carbonated liquid spray travels from the nozzle attached to the stationary robotic arm to the object to be sprayed. However, because the release of the gas cools the temperature of the dip solution being sprayed and the escape of the gas from the dip solution with concomitant foaming is dependent on the temperature, these competing actions minimize the degree of foaming. The necessary energy required to transport the dip to the teat is supplied by the pressurization of the dip solution tank with the CO₂ gas. Unlike the liquid spray that required twice the amount of liquid or the air aspirated foam that failed to sufficiently contact the teat from a distance, the carbonated spray was able to reach the teat with only a 0.1 second spray valve opening.

However, as it is easier for a dissolved gas to be released from a warmer liquid than from a colder liquid, once the dip solution is in contact with a warm surface the dip solution temperature is increased and the dissolved CO₂ and/or N₂O are released and generate the foam.

Thus, when the carbonated dip solution comes into contact with the warm gloved finger simulation, the heat supplied by the warm-gloved finger simulation to the carbonated dip solution warms the temperature of the carbonated dip solution. As a result, on contact with a the warm surface, significant foaming can occur. Once the preparation has been sprayed, the nozzle can be re-located and the next teat can be treated. The foam is retained on and covers the gloved finger simulation.

### Example 2

### Use of CO₂ to Generate Foam

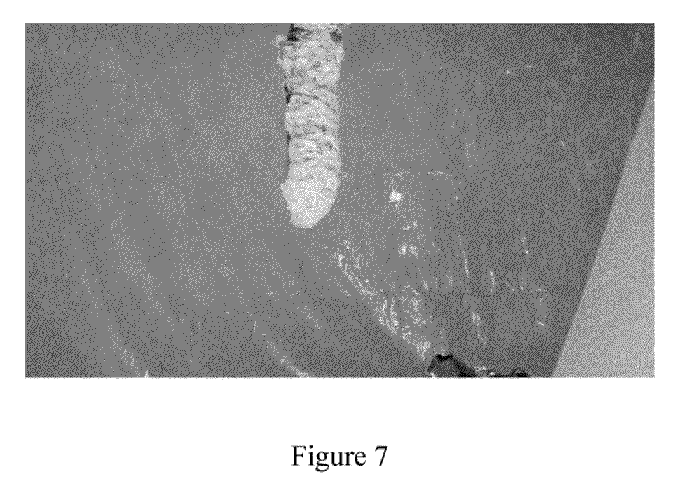

The system 100 (FIG. 3) used in Example 1 is tested with CO₂ as the propellant/diffusion gas in order to test the expanding spray. The resulting carbonated spray expands to approximately 3 mm in diameter in approximately 0.5 seconds, where the individual bubbles in the resulting foam varying in size from approximately 10 microns to approximately 100 microns. The foam bubbles coalesce in approximately 5-10 minutes (as shown in FIG. 7). After coalescing, the liquid drips from the teat and the remaining liquid dries on the teat.

The system (FIG. 3) was used with CO₂ as the propellant/diffusion gas using a variety of teat dip solutions from different manufacturers as well as different dip formulations from the same manufacturer. All dip solutions yielded similar results. Some produced different foam characteristics than others. In all of the dip solutions tested, the gas diffused foam gave better coverage, remained on the teat longer and used less dip solution or preparation compared to the liquid spraying of the dip solution.

### Example 3

### Use of N₂O Spray to Generate Foam

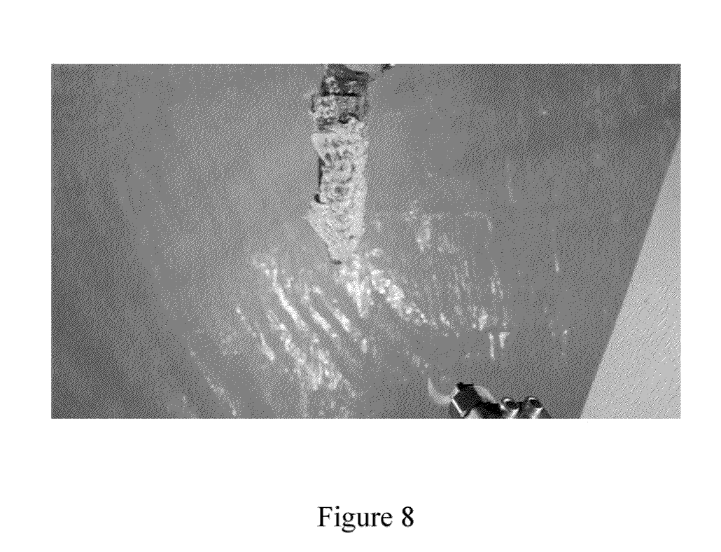

The system (FIG. 3) was used with nitrous oxide as the propellant/diffusion gas. In an embodiment of the invention, nitrous oxide (N₂O) can be used as a propellant/diffusion. N₂O is partially soluble in water, but is almost completely soluble in fats and oils (the skin conditioner provided in the cow dip). Under standard room temperature and 100 psi pressure, approximately four volumes of N₂O can be dissolved in one volume of the dip solution. The resulting nitrous oxide foam expands to approximately 6 mm in diameter, where the individual bubbles in the foam vary in size from approximately 1 micron to approximately 10 microns. These bubbles coalesce in approximately 10-15 minutes (as shown in FIG. 8). After coalescing, the remaining liquid dries on the teat. It is noted that the amount of N₂O that can be dissolved in one volume of dip solution will increase if more conditioners (containing one or more of fats, oils, fatty acids and triglycerides) are used since N₂O is almost completely soluble in fats and oils.

A number of differences were observed with the N₂O foam compared with the CO₂ foam. Firstly, the N₂O generated foam was more rigid or stable when it came into contact with an simulated cow teat, presumably because of the smaller bubble size and the bubble structures created by the expanded fat globules. Secondly, the textural sensation or “feel” of the N₂O foam was different from that of the CO₂ foam. The N₂O foam felt slicker as if it had more lubricant or a better lubrication capability than that of the CO₂ foam or the non-gas diffused dip solution. Thirdly, the N₂O foam had a longer life time than the CO₂ foam and thus took a lot longer to coalesce into a liquid than the CO₂ foam.

Under standard room temperature and pressure, the fat molecules in the dip solution are small and do not separate out of the dip solution, but rather form a colloidal suspension. When the dissolved N₂O escapes from the fat globules and liquid in the dip solution, the bubbles that are formed cause the fat globules that are now much smaller to begin to partially coalesce, but rather than separating out of the solution, the fat molecules form chains and clusters and adsorb to and spread around the N₂O bubbles. As the fat partially coalesces, it causes one fat-stabilized bubble to be linked to the next, and so on. In this manner, the N₂O reduces the size of the fat and oil globules in the dip solution and the resulting mixture takes on a smooth texture. This results from the formation of this partially coalesced fat structure stabilizing the bubbles. The water, iodine and other components of the dip solution are trapped in the spaces around the fat-stabilized bubbles.

When the dip spray is first turned on, the vessel pressure shoots the N₂O dip solution mixture out of the spray nozzle. Once ejected out of the nozzle, the pressure keeping the N₂O dissolved in the dip solution is reduced. As a result, the N₂O boils out of the dip mixture and in so doing the fat globule is expanded and broken into even smaller globules ultimately forming the foam. These smaller fat stabilized air bubbles have a stable three dimensional structure and therefore take longer to coalesce and turn back into liquid. The N₂O foam takes up a significantly greater volume (approximately 4 fold increase) than the original dip solution. The smaller fat globules created by the N₂O infusion increase the dispersion of the fats and oils in the dip solution giving the resulting foam, as indicated above, a more lubricated feel than non-N₂O diffused dip does. This results in better coverage and allows more of the skin conditioner to be absorbed into the teat tissue.

### Use of Combination of CO₂ and N₂O to Generate Foam

In various embodiments of the invention, mixtures of CO₂ and N₂O can be used as a propellant/diffusion gas for the dip solution preparation. The combination of CO₂ and N₂O can be advantageous compared to either CO₂ or N₂O gas alone or other gasses. CO₂ is able to expand aqueous solutions. However, when CO₂ is combined with N₂O, the bubble formation become smaller and the foam more structured. The CO₂ and N₂O combination results in a greater expansion of the dip than either gas alone. Further, N₂O in food grade form can be difficult to obtain and distribute because of concerns about its illegal use as an inhalant by people not licensed to administer anesthetics. In the State of California, the possession of N₂O with intent to inhale is a misdemeanor. Due to the health risks associated with carbon dioxide exposure, the U.S. Occupational Safety and Health Administration says that average exposure for healthy adults during an eight-hour work day should not exceed 0.5%. For short-term exposure, the U.S. National Institute for Occupational Safety and Health (NIOSH) and American Conference of Government Industrial Hygienists (ACGIH) limit is 3%. NIOSH also states that carbon dioxide concentrations exceeding 4% are immediately dangerous to life and health. Thus, adding CO₂ to N₂O reduces the risk that the mixture will be used as an inhalant. Distribution of the combined gasses are regulated the same as the distribution of straight CO₂ alone.

### Example 4

### Use of 90% CO₂ and 10% N₂O to Generate Foam

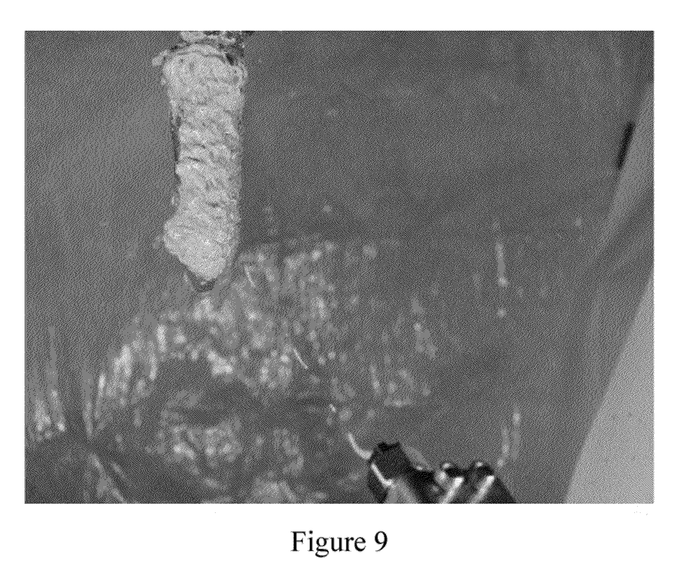

The system 100 (FIG. 3) was tested with approximately 90% carbon dioxide and approximately 10% nitrous oxide as the propellant/diffusion gas. Under standard room temperature and 100 psi pressure, approximately four volumes of dip solution can be dissolved per volume of propellant/diffusion gas. The approximately 90% carbon dioxide and approximately 10% nitrous oxide foam expands to give a foam with larger bubble size than the foam with higher N₂O compositions. Further, free liquid can be observed in addition to foam deposited on the gloved finger. Individual bubbles in the foam vary in size from 1 micron to 10 microns. These bubbles coalesce in approximately 10-15 minutes. After coalescing, the liquid drips from the teat. The remaining liquid dries on the teat. FIG. 9 shows a photograph of a foam formed using approximately 90% carbon dioxide and approximately 10% nitrous oxide as the propellant/diffusion gas directed onto a gloved finger.

### Example 5

### Use of 50% N₂O and 50% CO₂ to Generate Foam

In various embodiments of the invention, a mixture of approximately 50% CO₂ and approximately 50% N₂O was used as the propellant/diffusion gas in the dip solution. The increased nitrous oxide content is advantageous for use with dip solutions with a higher content of skin conditioner (or other hydrocarbon bound substances. Thus, the optimal percentage of nitrous oxide in the dip solution will depend on the amount of skin conditioner included in a particular dip solution.

This foam that was different from the foam generated with 90% CO₂ and 10% N₂O or the foam generated with 90% N₂O and 10% CO₂ combinations. No free liquid was observable and the bubble structure stayed intact longer, where foam can be observed up to 30 seconds after the spray was applied. The viscosity of the liquid passing through the spray nozzle was also observed to increase to a point that to deliver the same quantity of liquid dip the pressure of the gas needed to be increased to 150 psi. The spray valve applied approximately 120 mL of dip solution using 50% CO₂ and 50% N₂O. In comparison, using gas combinations other than 50% CO₂ and 50% N₂O or the gasses individually, approximately 133 mL of dip solution was applied using the same valve and time. This is due to the impact of the viscosity change as the dip solution passes through the nozzle restriction caused by the interaction of the N₂O with the skin conditioning in the cow teat dip.

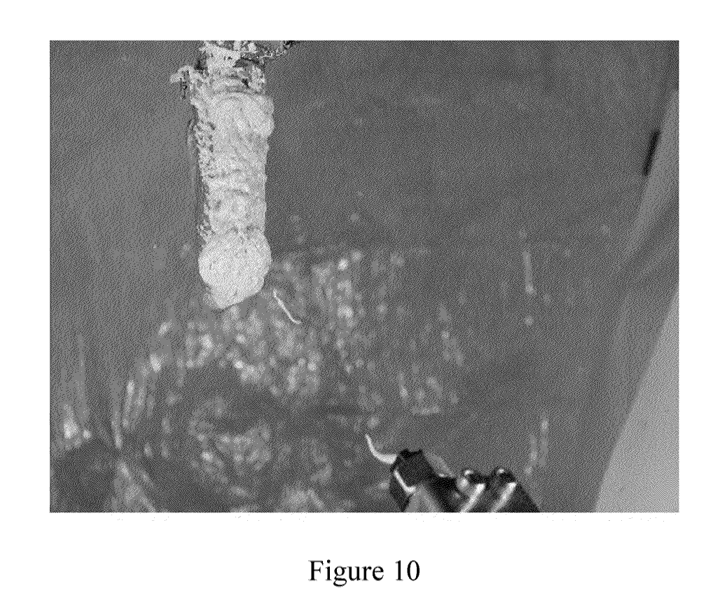

FIG. 10 shows a photograph of a foam formed using approximately 50% carbon dioxide and approximately 50% nitrous oxide as the propellant/diffusion gas directed onto a gloved finger. It should be noted that when using the iodine based dip, the color of the foam can be used as an indicator of the degree of expansion of the foam. That is, the lighter the color of the foam the greater the expansion of the liquid. Color change as the foam collapses can be used as a good indication of the speed the collapse occurs. With other propellant/diffusion gasses the free liquid can be identified by the dark reddish brown lines that form while the foam is collapsing. The 50% N₂O and 50% CO₂ foam was very uniform during collapse and had no lines.

### Example 6

### Use of 90% N₂O and 10% CO₂ to Generate Foam

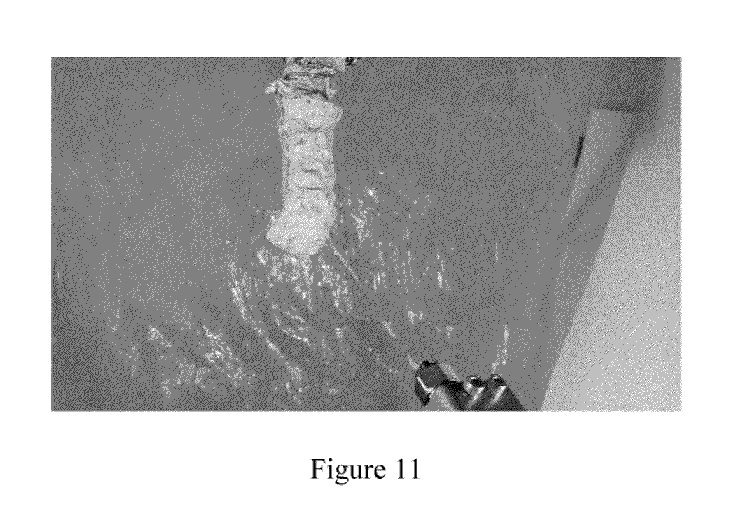

In various embodiments of the invention, a mixture of approximately 90% N₂O and 10% CO₂ was used as a propellant/diffusion gas in the dip solution. The increased nitrous oxide content produced foam that had very small (shaving cream like) bubble structure but it can be observed that there is some free liquid dip that was not affected by the gas. FIG. 11 shows a photograph of a foam formed using approximately 90% N₂O and 10% CO₂ as the propellant/diffusion gas directed onto a gloved finger simulation.

### Comparison of Examples 4, 5 and 6

Between examples 4, 5 and 6, the 50% N₂O and 50% CO₂ foam produced the lightest colored foam and took the longest to turn back into a liquid, indicating the most dissolved gas per volume of liquid. Both the 90% CO₂ and 10% N₂O foam and the 90% N₂O and 10% CO₂ foam had a lower viscosity traveling through the nozzle as they both had slightly more material pass through the same orifice with the same shot time. The 50% N₂O and 50% CO₂ foam had the thickest most dense foam on initial contact for the liquid dip preparation used. The 90% N₂O and 10% CO₂ foam was next in dissolved gas and the 90% CO₂ and 10% N₂O foam was the least based on color. The dip preparation had a high percentage of skin conditioners, which contributed to the increased effectiveness of the higher concentration N₂O solution.

### Generation of Foam From the Liquid Cow Dip at the Teat

In an embodiment of the invention, between approximately 59% and 99% of the dip solution liquid transferred into the foam form is applied at the teat. In an alternative embodiment of the invention, between approximately 89% and 99% of the dip solution liquid transferred into the foam form is applied at the teat. In an embodiment of the invention, between approximately 1% and 10% of the dip solution can be collected after coalescing and falling from the teat. In an alternative embodiment of the invention, between approximately 0.1% and 1% of the dip solution can be collected after coalescing and falling from the teat. In various embodiments of the invention, between 49% and 98% of the teat solution can be available to treat the teat. This substantially, reduces the amount of dip solution that is wasted, compared with non-pressurized dip solution that is either sprayed, but does not contact the teat, or is used without dissolved gasses in a cup in order to dip the teat, but is left in the cup and is not applied to the teat and thus is wasted. In addition, when forming foam which is held in a cup to which the teat is then contacted, much of the foam does not transfer from the cup to the teat, resulting in wasted dip solution. Further, because of the ability of the spray to be retained on the teat, there is less of the teat dip solution that falls from the teat and is wasted. No other method of application provides this efficiency of use of the dip solution. Once the required amount of dip solution has been ejected from the nozzle, and the spray has contacted the teat, the nozzle can be arranged proximal to the next teat and another aliquot can be ejected. In this manner, no left over dip solution is wasted in the process.

### Manual Techniques

In various embodiments of the invention, an approximately 50% CO₂ and approximately 50% N₂O propellant/diffusion gas formed dip mixture can be used in manual techniques for applying teat dip solution. Existing dip application equipment and non-robotic equipment can be modified to take advantage of this invention. New manual dip application installations can also be designed to have means for applying the propellant/diffusion gas diffused dip preparation. In various embodiments of the invention, an approximately 50% CO₂ and approximately 50% N₂O propellant/diffusion gas diffused dip mixture can be used in techniques that apply dip using dip cups, as well as manually controlled spraying devices.

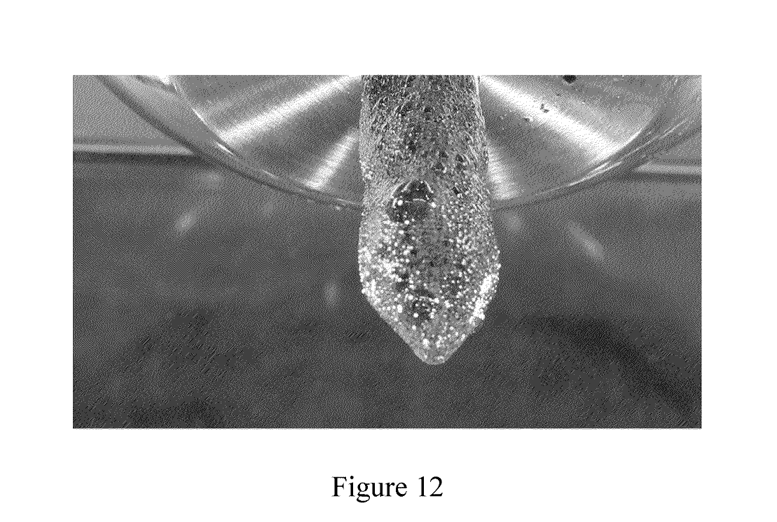

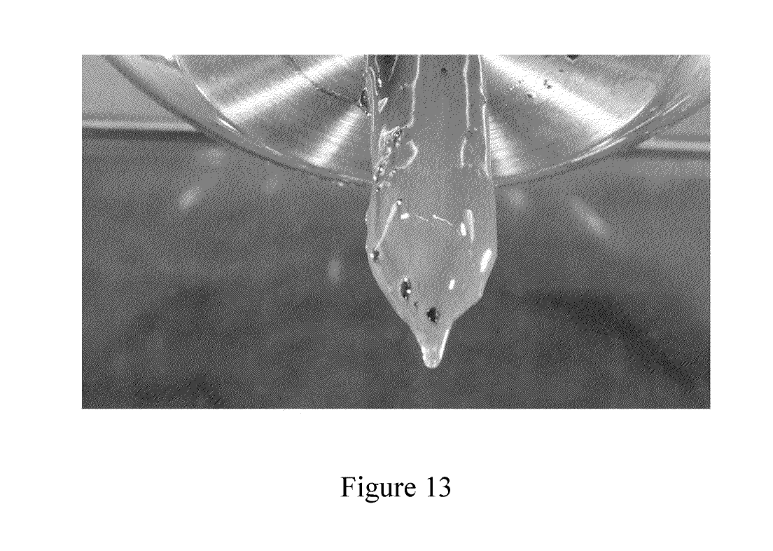

FIG. 12 illustrates a gloved finger simulation wherein air aspirated foam was manually applied to the gloved finger using a dip cup. FIG. 13 illustrates a gloved finger simulation wherein a 50% N₂O and 50% CO₂ foam was manually applied to the gloved finger simulation using a dip cup. As is apparent when comparing the two figures, the 50% N₂O and 50% CO₂ foam provides much better and complete coverage of the same gloved finger simulation than the air aspirated foam. In fact, it was found that the air aspirated foam had bubbles that were up to thousands of times bigger than the bubbles for the 50% N₂O and 50% CO₂ foam. Furthermore, it was found that the air aspirated foam actually wicks sanitization material away from the gloved finger simulation as shown by the very large bubble patterns left on the gloved finger simulation, thus leaving less dip preparation in contact with the gloved finger simulation. Conversely, the 50% N₂O and 50% CO₂ preparation generated foam micro bubble structure caused more sanitization material to stay in contact with the gloved finger.

### Non-Iodine Disinfectants

The CO₂ and N₂O propellant/diffusion gas has been used with a non-iodine disinfectants and sanitizing agents, and found to be suitable. The CO₂ and N₂O propellant/diffusion gas mixture can be adjusted in terms of gas components and concentration of each gas to any composition of sanitizing agent, skin conditioner, water or other carrier phase.

Increased Foam with Increased Heat

In an embodiment of the invention, when the spray comes into contact with a body part or surface of an animal, the increase in temperature of the gas diffused dip solution results in a significant increase in the foaming of the gas diffused dip solution. In an embodiment of the invention, when the spray exits the nozzle, it expands approximately 10%, whereas when the spray comes into contact with the cow's teat, the spray expands the remaining approximately 90% generating the foam depicted. Since an increase in temperature of the gas diffused dip solution results in increased foaming, it may be desirable to increase the ambient temperatures outside the nozzle to promote better foaming, especially if the spray is being applied in cold environments. In an embodiment, a semiconductor light source may be used as the heat source thereby illuminating the expanding spray and increasing the temperature thereby causing the expanding spray to further expand. In another embodiment, a heat or light source can also be applied to the cow's teats to assist in foaming of the spray upon contact. In yet another embodiment, a heat or light source, including an infra red, visible or ultra violet light source, such as, for example, a laser source, can also be directed at the expanding spray and heat the spray in flight before it comes in contact with a surface as a means of further increasing the rate of foaming and/or heat the spray as the spray contacts the animal, and/or heat the portion of the animal that the spray is applied to.

### Other Gases

Other gases or combination of gasses with similar characteristics to CO₂ and N₂O can be employed, within the spirit and scope of the invention. These other gases may include, but are not limited to, helium and nitrogen. Other embodiments of sanitizing solutions, can be understood by a person of ordinary skill in the art after a review of the specification, the claims, and the figures. Other gases or combination of gasses with similar characteristics to CO₂ and N₂O can be employed, within the spirit and scope of the invention. These other gases may include, but are not limited to, helium, nitrogen, isobutane, butane, propane, butyne, acetylene, dimethylether, triflouromethane and vinyl chloride. Other embodiments of sanitizing solutions, can be understood by a person of ordinary skill in the art after a review of the specification, the claims, and the figures.

### Foaming Agents

In an embodiment, the fats/oils may be substituted with foaming agents that can include, but are not limited to, myristic acid, steric acid, triethanolamine, sodium lauryl ether sulfate (SLES), sodium lauryl sulfate (SDS) and ammonium lauryl sulfate (ALS). In another embodiment, the spray foam can include a combination of fats/oils (e.g., glycerin, hydrogenated vegetable oil) in addition to other foaming agents.

### Other Delivery Systems

The gas diffused dip solution disclosed herein is not limited to being stored and/or delivered by the system 100 illustrated in FIG. 3. In an embodiment, the spray can be stored and delivered using portable devices, such as backpack units, hand-held units, and wheeled units that can fit through standard doorways. Additionally, a dispensing system can be mounted on a mobile platform such as a self-propelled cart or on a cart that is pulled or pushed by a motorized transport or is manually pulled or pushed.

### Charging Station

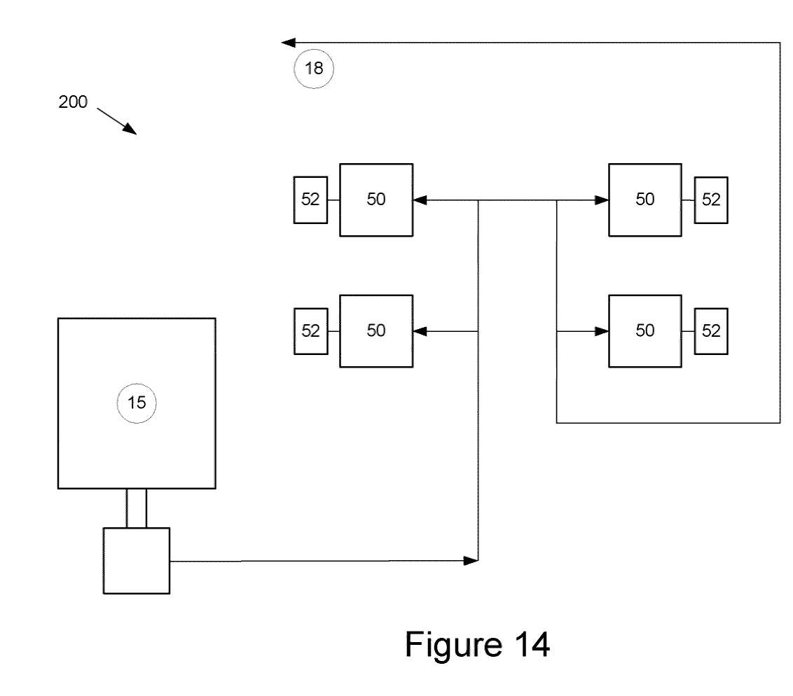

In an embodiment of the invention, the apparatus illustrated in FIG. 3 can be adapted to be used as a charging station system 200 for filling fixed storage tanks or portable dispensing spray containers with the gas diffused dip solution. In this embodiment, the spray valves 24 and nozzles 16 in FIG. 3 can be replaced with charging stations 50 having coupling devices. The coupling devices can be used to engage the charging stations 50 to portable hand-held spray container 52 as shown in FIG. 14. It is noted that FIG. 14 illustrates the portion of the apparatus shown in FIG. 3 having the recirculation pump 15 and the recirculation loop 18. In an embodiment, the charging stations 50 and the spray container 52 can include two mating halves of a self-sealing quick disconnect coupling. In this assembly, each half contains a valve that is held open when the coupling is connected. When the coupling is open, it allows fluid to flow through the coupling. The recirculation pump 15 can provide the motive force to send the gas-diffused dip into the spray container 52. When the coupling is disconnected, a spring in each half closes the valve, preventing the loss of fluid and the entrance of air. It is to be understood that other coupling device on the charging stations 50 could be used and still fall within the spirit and scope of the invention.

In an embodiment, a portable and/or hand-held spray container 52 can have its own spray valve and nozzle for delivering the gas diffused dip solution. In another embodiment, the spray container may be installed into another separate unit, for example, a backpack unit, that includes its own separate means of delivering the dip solution that may include the use of spray valves and nozzles. Regardless of the specific system employed, the spray container 52 can be continually recharged after its contents have been depleted.

### Hand-Held Units

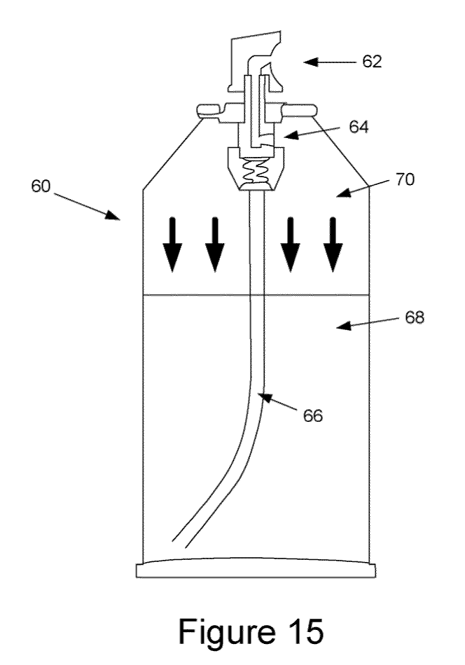

In another embodiment, the gas-diffused dip solutions can be placed in smaller disposable hand held units. For example, a gas-diffused dip solution can be placed in an aerosol can. A cross-sectional view of an aerosol can 60 in accordance with an embodiment of the invention is illustrated in FIG. 15. The aerosol can 60 can include a nozzle 62, seal 64, and dip tub 66 as found in most standard aerosol cans. In an embodiment, the aerosol can 60 includes the gas-diffused dip solution 68 and pressurized gas 70. As with the gas-diffused dip solutions described above, the pressurized gas 70 can include any gas including, but not limited to, CO₂, N₂O, helium and nitrogen.

### Embodiments of the Invention for Use in a Broad Range of Industries

The embodiments of the invention set forth above relate to gas diffused teat dip solutions used in the dairy industry or other animal industries. However, gas diffused sanitizers and disinfectants wherein the gas can be liberated at a controlled event for the purpose of generating useful foam can have many applications outside of the diary industry.

In an embodiment, gas diffused foams can be used to sanitize and disinfect any inert surface and/or object found in food processing facilities (e.g., meat packing plants), food service facilities (e.g., restaurants, cafeterias), healthcare facilities (e.g., hospitals, surgery centers, clinics, long-term care), hospitality facilities (e.g., hotels, motels, resorts), educational facilities (e.g., schools, universities), and commercial facilities (e.g., office buildings, shopping malls, retail stores).

In an embodiment, the preparation can include a gas diffused aqueous solution having a sanitizing agent and other fats/oils (e.g., glycerin). As used with the teat dips, the gas that is diffused with the aqueous solution may include N₂O and/or CO₂. Other gasses may also be used instead of N₂O and CO₂ that include, but are not limited to, helium and nitrogen. These gasses can be used alone, or in combination with one another. In an embodiment, the preparation can include a gas diffused aqueous solution having a sanitizing agent and other fats/oils (e.g., glycerin). As used with the teat dips, the gas that is diffused with the aqueous solution may include N²O and/or CO₂. Other gasses may also be used instead of N₂O and CO₂ that include, but are not limited to, helium, nitrogen, isobutane, butane, propane, butyne, acetylene, dimethylether, triflouromethane and vinyl chloride. These gasses can be used alone, or in combination with one another.

The sanitizing agent may include, but is not limited to, iodine, acidified sodium chlorite, sodium hypochlorite, chlorhexidine, hydrogen peroxide, caprylic/capric acid(s), bronopol, dodecyl benzene sulfonic acid (DDBSA), nisin, glycerol monolaurate, quaternary ammonium, acetone, as well as other alcohols, acetone, aldehydes, oxidinzing agents, phenolics, quaternary ammonium compounds, polyaminopropyl biguanide and other hydrocarbon-based solvents. Hydrocarbon based solvents could also be used in combination with N₂O, since N₂O is particularly soluble in hydrocarbon solvents.

In an embodiment, the fats/oils may be substituted with foaming agents that can include, but are not limited to, myristic acid, steric acid, triethanolamine, sodium (amyl ether sulfate (SLES), sodium lauryl sulfate (SDS) and ammonium lauryl sulfate (ALS). In another embodiment, the spray foam can include a combination Of fats/oils in addition to other foaming agents.

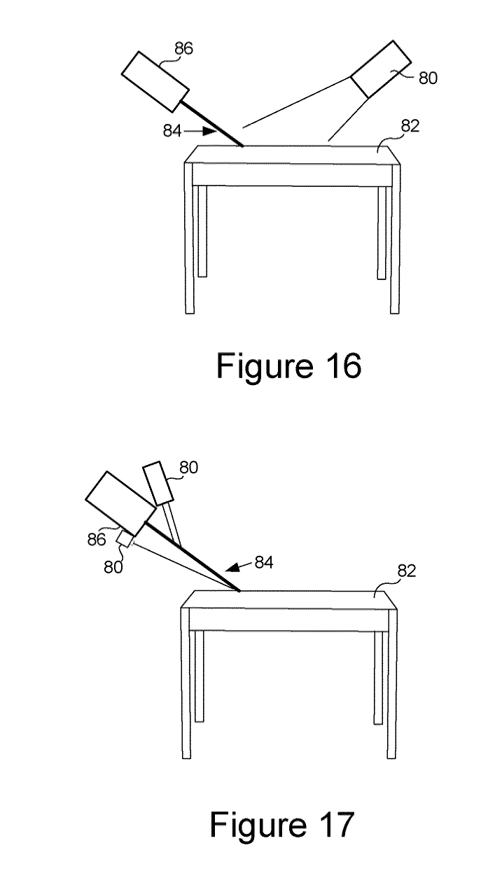

In an embodiment of the invention, a propellant/diffusion gas can be used to dispense the sanitizing spray when the surface being contacted is inert. As with the dip solution described above, the sanitizing spray used on inert surfaces can be delivered primarily as a liquid and then expand to form a foam upon contact with the inert surface. In such a situation, the spray expansion will be dependent on the temperature of the solution prior to ejection and the temperature of the inert surface. Typically the dip solution will be kept at approximately 273-288° K. If the inert surface has a temperature significantly above 288° K. (e.g., above 289° K.) then the spray can foam, but the process can take longer to deplete the spray of dissolved gas. Alternatively, the ambient temperature outside the nozzle or the inert surface temperature can be adjusted by using a heat source directed on the expanding spray formed at the nozzle or at the inert surface. In an embodiment, a semiconductor light source 80 may be used as the heat source thereby illuminating the expanding spray and increasing the temperature thereby causing the expanding spray to further expand into foam. In another embodiment, a heat or light source 80 can be applied to the inert surface 82 to assist in foaming of the spray 84 as shown in FIG. 16. In yet another embodiment, a heat or light source 80, which can include an infra red, visible or ultraviolet light source, such as a laser light source, can also be directed at the expanding spray proximal to the nozzle 86 and heat the spray 84 in flight before it comes in contact with a surface 82 or as the spray contacts the surface as a means of further increasing the rate of foaming as shown in FIG. 17.

As with the spray teat dip solutions, spray for use in non-dairy industries can be stored and delivered using portable devices, such as backpack units, wheeled units that can fit through standard doorways, or even aerosol cans. In another embodiment, docking stations may be employed that can charge smaller hand held units for ease of use (see FIG. 14).

### Fire Fighting Spray Foam

The use of foaming agents to increase the effectiveness of water in the prevention and suppression of fires is well known. Fire fighting foams can include, but are not limited to, Class A foams for fighting wildfires, Class B foams for use with flammable liquids, synthetic foams such as aqueous film forming foams (AFFF) and alcohol-resistant aqueous film forming foams (AR-AFFF), and protein foams such as regular protein foam (P), fluoroprotein foam (FP), alcohol resistant fluoroprotein foam (AR-FP), film forming fluoroprotein (FFFP), and alcohol-resistant film forming fluoroprotein (AR-FFFP).

A compressed air foam system (CAFS) is a system used by firefighters to deliver the fire retardant foam. Examples of existing foam generating systems can be found in U.S. Pat. No. 6,357,532 to Laskaris et al., entitled “Compressed Air Foam Systems,” and U.S. Pat. No. 6,733,004 to Crawley entitled “Apparatus for Generating Foam.”

In an embodiment of the invention, the same principles as used for the cow dip spray can be used in the fire fighting context. Accordingly, water, a fire retardant foam, and a gas (as opposed to air) can be used to form a gas-diffused fire fighting spray. In an embodiment, CO₂ can be used as the gas. Other embodiments may use other gases such as helium and nitrogen.

Using a gas-diffused foam is advantageous since it delays foaming as compared to an air aspirated foam. Specifically, with a gas-diffused spray, the foaming is substantially delayed until the spray contacts the object it was directed at and heat is added. This allows the spray to travel primarily as a liquid stream, which increases the distance that the spray will travel from the nozzle as compared to an air aspirated spray that tends to foam more readily before contacting the intended target. Once the gas-diffused spray reaches its intended target, the heat from the fire on or near the object will result in significant foaming on the object. The foam will also continue to grow after contact as more gasses are released. Using a gas-diffused spray also assists in excluding oxygen from a fire making it a better barrier than an air aspirated foam.

In an embodiment, water, gas and a fire retardant foam concentrate may be batch mixed and stored prior to use. In another embodiment, gas diffused water can be stored in a tank, and then combined with the fire retardant foam concentrate in a mixing chamber immediately before use. This may be beneficial if the fire retardant foam concentrate readily breaks down or deteriorates upon being introduced to water (as is the case with Class A foam). The gas diffused spray may be stored and dispensed using large machinery, such as a CAFS loaded onto a motor vehicle, or smaller devices like a portable backpack or even a standard fire extinguisher. Any other methods and devices can be used to actually deliver the gas-diffused spray as would be known to one having ordinary skill in the art and still fall within the scope of this invention.

While embodiments and applications of this invention have been shown and described, it would be apparent to those skilled in the art having the benefit of this disclosure, that many more modifications than mentioned above are possible without departing from the inventive concepts herein.

What is claimed is:

1. A method of treating an animal comprising the steps of:
applying a preparation which includes an aqueous solution to the animal, wherein the aqueous solution includes (1) at least one of a sanitizing agent and a disinfecting agent, (2) a conditioner and (3) a diffusible gas, wherein the diffusible gas is at least partially dissolved in the aqueous solution;
allowing the animal to warm the preparation; and
whereby the diffusible gas is liberated from the aqueous solution to generate a foam as the animal warms the aqueous solution.

2. The method of claim 1 wherein:
the applying step includes applying a conditioner which includes at least one of fats, oils, fatty acids, and triglycerides and applying a diffusible gas including at least one of carbon dioxide, nitrous oxide, and a mixture of carbon dioxide and nitrous oxide.

3. The method of claim 1 wherein the teats of a mammal are treated wherein:
the applying step includes spraying the aqueous solution as a liquid onto the teats of the mammal; and
the allowing step includes allowing the aqueous solution to be warmed by the teats of the mammal in order to liberate the diffusible gas from the aqueous solution to generate a foam on the teats of the mammal as the aqueous solution warms.

4. The method of claim 3 wherein:
the applying step includes applying a conditioner which includes at least one of fats, oils, fatty acids, and triglycerides and applying a diffusible gas including at least one of carbon dioxide, nitrous oxide, and a mixture of carbon dioxide and nitrous oxide.

5. The method of claim 1 including the step of preventing mastitis in the teats of a mammal.

6. The method of claim 1 wherein said aqueous solution is delivered separately from the diffusible gas to the site of treatment of the animal and the applying step includes at the site of treatment of the animal allowing the diffusible gas to at least partially dissolved in the aqueous solution.

7. The method of claim 1 wherein said diffusible gas is delivered as a liquid to the site of treatment of the animal and the applying step includes allowing the diffusible gas to at least partially dissolved in the aqueous solution at the site of treatment of the animal.

8. The method of claim 7 wherein the applying step includes said liquid as a mixture of about 50% carbon dioxide and about 50% nitrous oxide.

9. The method of claim 1 wherein the applying step includes said diffusible gas as a mixture of about 50% carbon dioxide and about 50% nitrous oxide.

10. The method of claim 1 wherein said preparation is applied to the animal mostly as a liquid and forms a foam on the animal.

11. The method of claim 1 wherein applying step includes using a conditioner that includes a fat and a diffusible gas that includes nitrous oxide and said foam includes fat stabilized gas bubbles.

12. The method of claim 1 wherein the applying step includes allowing said diffusible gas to diffuse in said aqueous solution, wherein said diffusible gas is liberated from said preparation at a controlled event to generate a foam to treat the animal.

13. The method of claim 1 including applying heat from a heat source to at least one of the animal and the preparation to generate a foam to treat the animal.

14. The method of claim 1 wherein the applying step includes using a sanitizing agent that is at least iodine.

15. The method of claim 1 wherein said applying step uses said diffusible gas as a propellant gas for the delivery of the preparation to an animal.

16. The method of claim 1 wherein said applying step uses said diffusible gas with a water soluble component and a component that solvates the skin conditioner.

17. The method of claim 1 wherein the applying step uses said diffusible gas that is at least one of:
carbon dioxide;
nitrous oxide;
a mixture of carbon dioxide and nitrous oxide;
nitrogen;
helium;
a mixture of nitrogen and helium.

18. A method for treating an animal comprising the steps of:
applying a preparation to the animal, which preparation includes an aqueous solution including:
at least one of a sanitizing agent and a disinfecting agent;
a skin conditioner; and
a diffusible gas, wherein said diffusible gas is at least partially dissolvable in the aqueous solution, wherein the diffusible gas includes a water soluble component and a component that solvates the skin conditioner;
allowing the animal to warm the aqueous solution; and
whereby the diffusible gas is liberated from the aqueous solution to generate a foam as the animal warms the aqueous solution.

19. The method of claim 18 wherein said applying step uses a preparation including
said skin conditioner having at least one or more compounds selected from the group consisting of fats, oils, fatty acids, and triglycerides; and
said diffusible gas having one or more of:
carbon dioxide;
nitrous oxide; and
a mixture of carbon dioxide and nitrous oxide.

20. The method of claim 18 wherein the applying step uses said skin conditioner that includes a fat and said diffusible gas that includes nitrous oxide and said applying step generates said foam that includes fat stabilized gas bubbles.

21. The method of claim 18 wherein said applying step uses a preparation wherein:
(a) the diffusible gas includes carbon dioxide and nitrous oxide;
(b) the carbon dioxide is between a lower limit of about 5% and an upper limit of about 90% of the diffusible gas;
(c) the nitrous oxide is between a lower limit about 5% and an upper limit of about 90% of the diffusible gas; and
(d) the sanitizing agent is iodine, where the iodine is in the aqueous solution at a concentration between a lower limit of about 0.1% and an upper limit of about 20%.

22. A method for using a preparation to treat an animal comprising the steps of:
applying a preparation that can delivered as a foam having fat stabilized bubbles to treat an animal comprising:
an aqueous solution having at least a sanitizing agent including iodine;
a conditioner including one or more selected from the group consisting of fats, oils, fatty acids, and triglycerides; and
a diffusible gas, wherein the gas is partially dissolvable in the aqueous solution;
wherein the diffusible gas includes a water soluble component and a component that solvates the conditioner, and wherein the diffusible gas includes nitrous oxide;
allowing the animal to warm the preparation; and
whereby the diffusible gas is liberated from the aqueous solution to generate foam as the animal warms the aqueous solution.

23. A method for making a preparation for treating an animal comprising:
delivering an aqueous solution having at least one of a sanitizing agent and a disinfecting agent, and having a skin conditioner; and
delivering a diffusible gas, wherein said diffusible gas has the characteristic that the diffusible gas is at least partially dissolvable in the aqueous solution; and
wherein said diffusible gas is at least one of:
carbon dioxide;
nitrous oxide;
a mixture of carbon dioxide and nitrous oxide.

24. The method of claim 26 wherein the step of delivering the diffusible gas includes delivering the diffusible gas to the site of making the preparation as a liquid or as a gas.
